(19) United States
(12) Patent Application Publication
(10) Pub. No.: US 2026/0248377 A1
(43) Pub. Date: Aug. 27, 2026

(54) ILLUMINATOR SYSTEMS

(71) Applicant: ALCON INC., Fribourg (CH)

(72) Inventors: Lingfeng YU, Rancho Santa Margarita, CA (US); Steve X. CHEN, San Diego, CA (US); Ronald T. SMITH, Irvine, CA (US); Mark Harrison FARLEY, Laguna Hills, CA (US); Mikhail OVCHINNIKOV, Trabuco Canyon, CA (US); Pemba Tsering LAMA, Orange, CA (US); John PARK, Irvine, CA (US)

(21) Appl. No.: 19/542,839

(22) Filed: Feb. 18, 2026

Related U.S. Application Data
(60) Provisional application No. 63/762,840, filed on Feb. 25, 2025.

(51) Int. Cl. A61B 3/00; A61B 3/13

(52) U.S. Cl. CPC A61B 3/0008; A61B 3/13

(57) ABSTRACT

In certain embodiments, an illuminator system provides illumination for a patient interface. The illuminator system includes input bundles, a fiber randomizer, and output bundles. The input bundles include a first input bundle and a second input bundle. The first input bundle includes first fibers, where each first fiber transmits a first light from a first light source. The second input bundle includes second fibers, where each second fiber transmits a second light from a second light source. The fiber randomizer receives the first fibers from the first input bundle, receives the second fibers from the second input bundle, and distributes the first and the second fibers among subsets of output fibers. Each subset of output fibers has at least one first fiber and at least one second fiber. Each output bundle includes a subset of output fibers and provides the first light and the second light to the patient interface.

### FIELD

Embodiments of the present disclosure relate to illuminator systems for ophthalmic devices.

### BACKGROUND

Ophthalmic surgery typically uses illumination to image the eye of a patient. In some cases, different wavelengths of illumination are used. For example, visible light may be used to generate images from a microscope, and infrared light may be used to generate images used to register or track the eye.

### SUMMARY

In one or more embodiments, an illuminator system provides illumination for a patient interface. The illuminator system includes input bundles, a fiber randomizer, and output bundles. The input bundles include a first input bundle and a second input bundle. The first input bundle includes first fibers, where each first fiber transmits a first light from a first light source. The second input bundle includes second fibers, where each second fiber transmits a second light from a second light source of a different wavelength than the first light source. Each subset of output fibers has at least one first fiber and at least one second fiber. Each output bundle includes a subset of output fibers and provides both the first light and the second light to the patient interface. The fiber randomizer optically couples the input bundles and the output bundles.

Embodiments may include zero, one, two, some, most, or more of the following.

The output bundles are arranged to provide the first light and the second light around a circumference of the patient interface.

At least one output bundle is coupled to an objective lens of an ophthalmic microscope.

At least one input bundle has a diameter in a range of 0.4 millimeters (mm) to 2 mm.

At least one output bundle has a diameter in a range of 0.4 millimeters (mm) to 2 mm.

The shape of an input end of the first input bundle is substantially equivalent to the shape of the first light source.

The size of an input end of the first input bundle is substantially equivalent to the size of the first light source.

The number of the output bundles is in a range of three to twelve bundles.

The first light comprises a visible light.

The second light comprises an infrared light.

The fiber randomizer has a randomizer input end and a randomizer output end. The randomizer input end has a first relative distribution of fibers comprising the first fibers arranged in the first input bundle and the second fibers arranged in the second input bundle. The randomizer output end has a second relative distribution of fibers arranged as the subsets of output fibers, where each subset of output fibers comprises at least one first fiber and at least one second fiber. The second relative distribution of fibers may include a randomized distribution of the first fibers and the second fibers. The second relative distribution of fibers may be created by interweaving the first fibers and the second fibers along a length of the fiber randomizer. A given subset of output fibers may include an approximately equal number of the first fibers and the second fibers. By way of example, an approximately equal number of the first fibers and the second fibers may include a quantity within 0.5%, within 1%, within 2%, within 3%, within 4%, within 5%, within 7%, within 10%, within 15%, within 20%, or within 25% of each other.

In one or more embodiments, an illuminator system provides illumination for a patient interface. The illuminator system includes illumination subsystems. An illumination subsystem includes input bundles, a fiber randomizer, and output bundles. The input bundles include a first input bundle and a second input bundle. The first input bundle includes first fibers, where each first fiber transmits a first light from a first light source. The second input bundle includes second fibers, where each second fiber transmits a second light from a second light source of a different wavelength than the first light source. Each subset of output fibers has at least one first fiber and at least one second fiber. Each output bundle includes a subset of output fibers. The illumination subsystems provide both the first light and the second light to the patient interface. The fiber randomizer optically couples the input bundles and the output bundles.

Embodiments may include zero, one, two, some, most, or more of the following.

The illumination subsystems are arranged to provide the first light and the second light around a circumference of the patient interface.

At least one output bundle is coupled to an objective lens of an ophthalmic microscope.

At least one input bundle has a diameter in a range of 0.4 millimeters (mm) to 2 mm.

At least one output bundle has a diameter in a range of 0.4 millimeters (mm) to 2 mm.

The shape of an input end of the first input bundle is substantially equivalent to the shape of the first light source.

The size of an input end of the first input bundle is substantially equivalent to the size of the first light source.

The number of the output bundles is in a range of three to twelve bundles.

The first light comprises a visible light.

The second light comprises an infrared light.

The fiber randomizer has a randomizer input end and a randomizer output end. The randomizer input end has a first relative distribution of fibers comprising the first fibers arranged in the first input bundle and the second fibers arranged in the second input bundle. The randomizer output end has a second relative distribution of fibers arranged as the subsets of output fibers, where each subset of output fibers comprises at least one first fiber and at least one second fiber.

In one or more embodiments, an illuminator system provides illumination for a patient interface. The illuminator system includes a light engine and output bundles. The light engine generates a first light using a first light source, generates a second light using a second light source, splits the first light into first light portions, and splits the second light into second light portions. Each output bundle receives a first light portion, receives a second light portion, and provides the first light portion and the second light portion to illuminate the patient interface.

Embodiments may include zero, one, two, some, most, or more of the following.

The output bundles are arranged to provide the first light and the second light around a circumference of the patient interface.

At least one output bundle is coupled to an objective lens of an ophthalmic microscope.

### BRIEF DESCRIPTION OF THE DRAWINGS

FIG. 1 illustrates an example of an ophthalmic system with an illumination system that can direct light towards a patient interface in order to illuminate an eye, according to at least one embodiment described in the present disclosure;

FIG. 2 illustrates an example of a computing system, according to at least one embodiment described in the present disclosure;

FIGS. 3A through 3C illustrate an example of a system that includes an illumination system that directs illumination towards a patient interface to illuminate an eye, according to at least one embodiment described in the present disclosure;

FIGS. 4A through 4D illustrate an example of an illumination system with a fiber randomizer, according to at least one embodiment described in the present disclosure;

FIGS. 5A through 5C illustrate additional examples of illumination systems that include a fiber randomizer, according to at least one embodiment described in the present disclosure;

FIGS. 6A through 6C illustrate examples of input ends that receive light from light source systems, according to at least one embodiment described in the present disclosure; and

FIG. 7 illustrates an example of an illumination system with a light engine, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Ophthalmic surgery may use light of different wavelengths to provide illumination to image the eye of a patient. However, illumination systems that provide light from different light sources can be complicated and bulky. Accordingly, the present disclosure relates to illumination systems that provide light to a patient interface in order to illuminate an eye.

Certain embodiments of the present disclosure may provide improvements over previous illumination systems. For example, one or more embodiments may include a fiber randomizer that may provide a more homogenized mixture of the different wavelengths of light for illumination by using randomized bundles of fibers. As another example, one or more embodiments may provide an illumination system that is more compact than previous illumination systems due to the use of the randomized bundles of fibers.

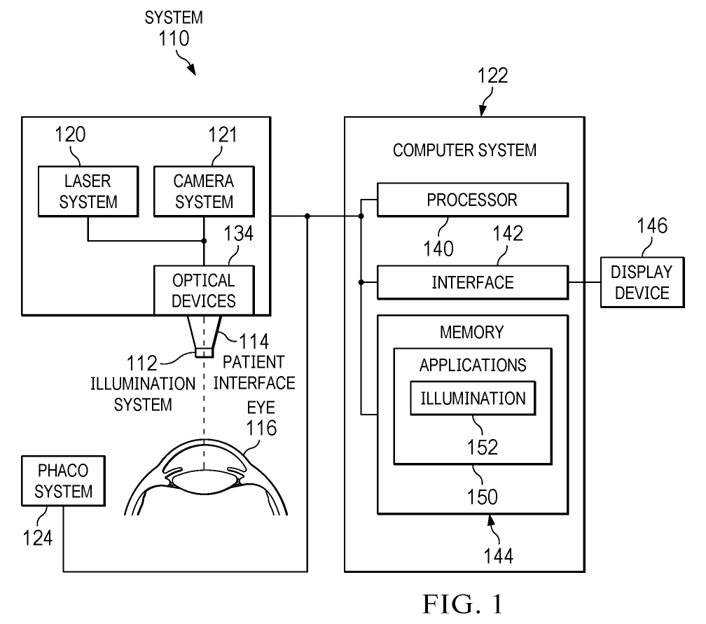

FIG. 1 illustrates an example of an ophthalmic system 110 with an illumination system 112 that can direct light towards a patient interface 114 in order to illuminate an eye 116, according to at least one embodiment described in the present disclosure. In the example, the ophthalmic system 110 includes the illumination system 112, a laser system 120, a camera system 121, a computer system 122, a phacoemulsification (phaco) system 124, one or more optical devices 134, and/or the patient interface 114, which may be coupled as shown. The computer system 122 includes a processor 140, an interface 142 (which may include a display device 146), and/or a memory 144, which may be coupled as shown. The memory 144 stores applications 150, such as an illumination application 152.

For ease of explanation, the embodiments are described using the following example xyz-coordinate system, which may be regarded as the coordinate system of the ophthalmic system 110, although any suitable coordinate system may be used. In the example, the z-axis is aligned with the optical axis of the laser system 120, and an xy-plane is orthogonal to the z-axis.

In one or more embodiments, the ophthalmic system 110 may be any suitable medical device that performs a medical procedure, such as a surgical and/or diagnostic procedure. In one or more embodiments, the ophthalmic system 110 may be a surgical system that directs a laser beam to a part of the eye (e.g., cornea, lens, and/or other eye tissue) to process the tissue (e.g., ablate and/or photodisrupt the tissue). In one or more embodiments, the ophthalmic system 110 may be a surgical system that performs cataract surgery (e.g., femtosecond laser assisted cataract (FLAC) surgery) to remove a crystalline lens from the eye. In FLAC surgery, a laser (e.g. a femtosecond laser) may be used to create photodisruptions in the crystalline lens arranged in a fragmentation pattern to segment the crystalline lens, in order to facilitate removal of the crystalline lens.

Turning to the components, the laser system 120 directs a laser beam towards the eye 116. The laser system 120 may include a laser source that generates the laser beam and a scanner that guides the laser beam. One or more optical devices 134 direct the laser beam towards the eye 116. The laser source may be a femtosecond laser or other ultrashort pulse laser. The laser beam may have any suitable pulse duration, such as in the order of nanoseconds, picoseconds, femtoseconds, or attoseconds. The laser beam may have any suitable wavelength, such as in the range of 150 nanometers (nm) to 20 micrometers (µm). Examples of ranges include the ultraviolet (e.g., in the range of 180 to 400 nm, such as 190 to 195 nm and/or 345 to 355 nm), visible, or infrared (including near-infrared) wavelength (e.g., in the range of 1000 to 1250 and/or 1250 to 1500 nm).

The camera system 121 includes one or more cameras that generates an image of the eye 116. A camera of the camera system 121 may be any suitable camera that captures and records images. For example, a camera may be a digital camera that records the image as digital image data. A digital camera may include: an image sensor that detects light reflected from an object, such as a digital image sensor (e.g., CCD or CMOS); an image processor that converts the sensor output to digital image data representing the image; and/or a memory that records the image as image data.

The phaco system 124 removes the lens from the eye 116. In one or more embodiments, the phaco system 124 uses ultrasonic energy to emulsify the lens and then suctions the lens from the eye 116. In other embodiments, the laser system 120 emulsifies the lens, and the phaco system 124 removes the lens from the eye 116 without the use of much, if any, ultrasonic energy.

The computer system 122 performs operations to illuminate the eye 116, including sending instructions to other components of the ophthalmic system 110 (e.g., the illumination system 112) to perform operations (e.g., direct illumination towards the eye 116). The computer system 122 may use applications 150 to perform the operations. For example, the illumination application 152 may be used to instruct the illumination system 112 to direct light from light sources to the patent interface 114 to illuminate the eye 116. The illumination system 112 is described in more detail herein.

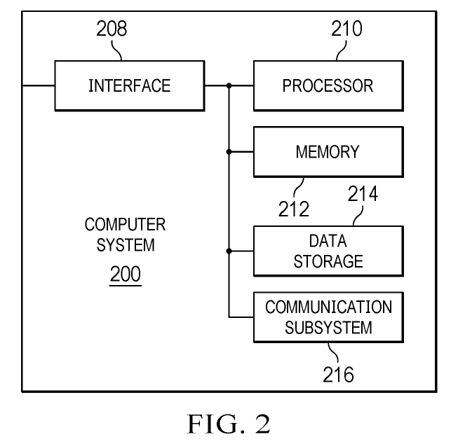

FIG. 2 illustrates an example of a computing system 200, according to at least one embodiment described in the present disclosure. The computing system 200 may include an interface 208, a processor 210, a memory 212, a data storage 214, and/or a communication subsystem 216, any or all of which may be communicatively coupled. Any or all of the computing system 200 may be implemented as computer hardware and/or software. One or more components of a computer system described herein may be implemented as described with reference to the computing system 200.

In the example, the interface 208 may receive input to the computing system 200 and/or send output from the computing system 200, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computing system 200. Examples of user interfaces include displays, Graphical User Interfaces (GUIs), touchscreens, foot pedals, keyboards, computer mouses (or mice), joysticks, gesture sensors, microphones, and speakers.

Generally, the processor 210 may include any suitable special-purpose or general-purpose computer, computing entity, or processing device including various computer hardware or software modules and may be configured to execute instructions stored on any applicable computer-readable storage media. For example, the processor 210 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 2, the processor 210 may include any number of processors distributed across any number of network or physical locations that are configured to perform individually or collectively any number of operations described in the present disclosure.

The processor 210 may perform any suitable operations. In some embodiments, the processor 210 may interpret and/or execute program instructions and/or process data stored in the memory 212, the data storage 24, or the memory 212 and the data storage 214. In some embodiments, the processor 210 may fetch program instructions from the data storage 214 and load the program instructions into the memory 212. After the program instructions are loaded into the memory 212, the processor 210 may execute the program instructions, such as instructions to perform any of the methods disclosed herein, respectively.

The memory 212 and the data storage 214 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 210.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 210 to perform a certain operation or group of operations.

The communication subsystem 216 may include any component, device, system, or combination thereof that is configured to transmit, receive, and/or otherwise exchange information over a network in order to communicate with any suitable entity, such as with other devices at other locations or at the same location or even within the same system. The communication subsystem 216 may provide for communication among the devices described in the present disclosure, communication networks, computing devices, and other systems. For example, the communication subsystem 216 may allow the system 200 to communicate with other systems, such as other computing devices and/or networks. In some embodiments, the communication subsystem 216 may include a modem, a network card (wireless or wired), an optical communication device, an infrared communication device, a wireless communication device (such as an antenna), and/or chipset. Examples of communication subsystem 216 include a Bluetooth device, an 802.6 device (e.g., that can communicate with a Metropolitan Area Network (MAN)), a WiFi device, a WiMax device, cellular communication facilities, and/or the like.

One skilled in the art will recognize that modifications, additions, or omissions may be made to the system 200 without departing from the scope of the present disclosure. For example, the system 200 may include more or fewer components than those explicitly illustrated and described.

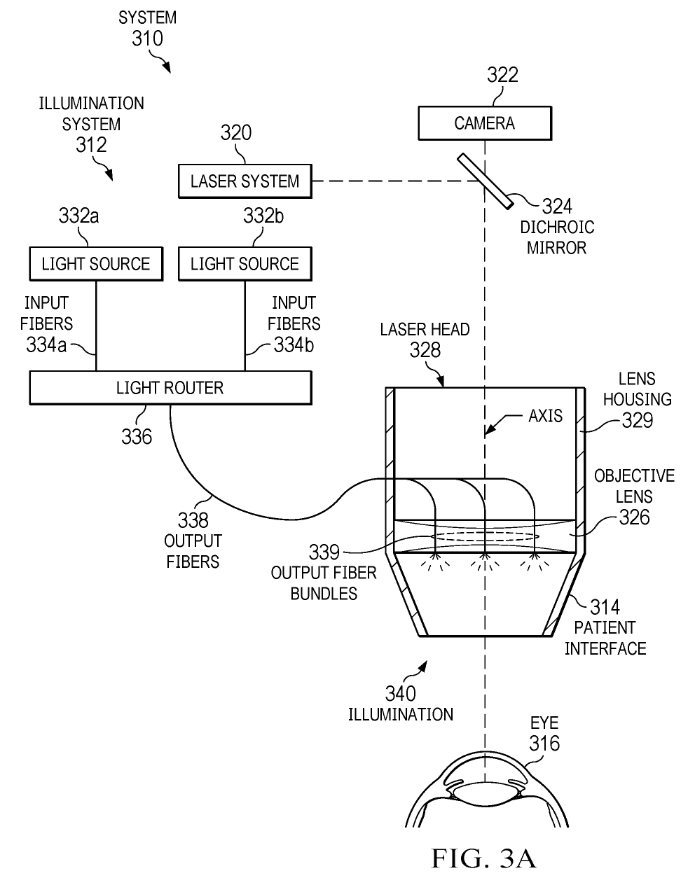

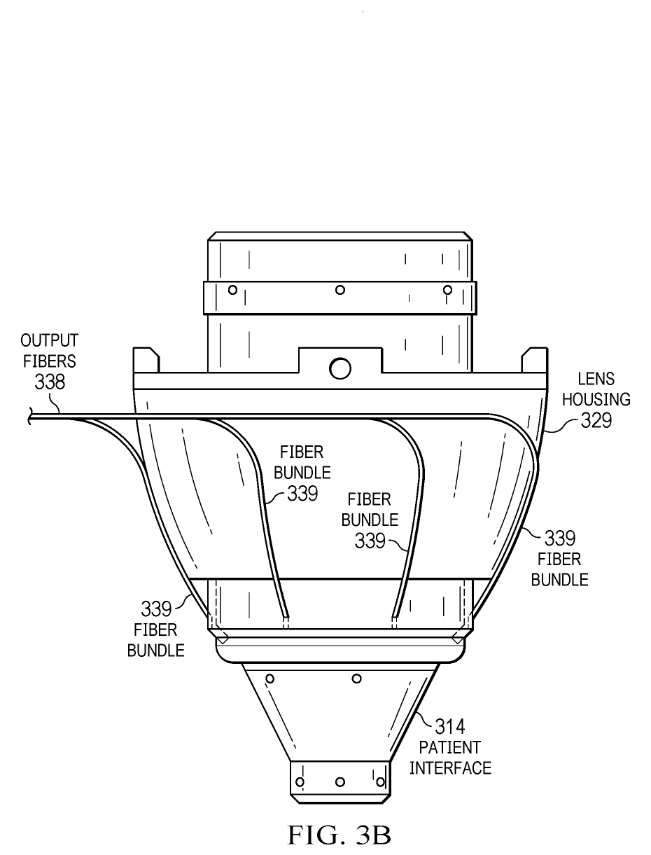

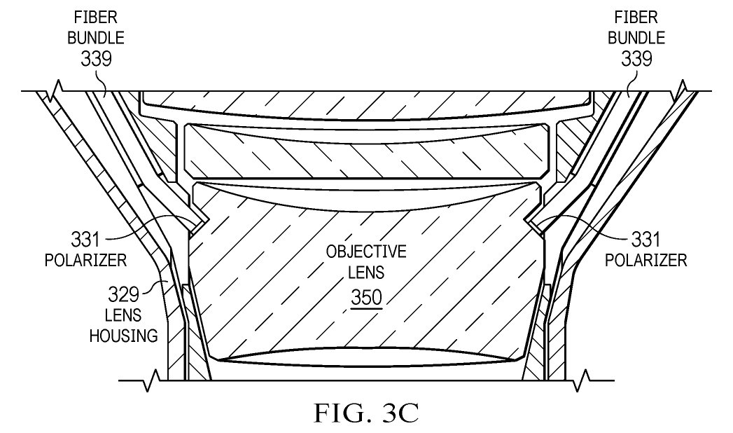

FIGS. 3A through 3C illustrate an example of a system 310 that includes an illumination system 312 that directs illumination 340 towards a patient interface 314 to illuminate an eye 316, according to at least one embodiment described in the present disclosure. FIG. 3A shows the system 310 with the illumination system 312, the patient interface 314, a laser system 320, a camera 322, a dichroic mirror 324, an objective lens 326, a laser head 328, and/or a lens housing 329, which may be coupled as shown. The illumination system 312 includes light sources 332 (332a and 332b), input fibers 334 (334a and 334b), a light router 336, output fibers 338, and/or output fiber bundles 339, which may be coupled as shown. FIG. 3B shows the patient interface 314 coupled to the lens housing 329. The output fiber bundles 339 are disposed within the interior of the lens housing 329 and arranged to provide illumination about the circumference of the patient interface 314. FIG. 3C shows an objective lens 350 (e.g., a distal objective lens of an ophthalmic microscope), disposed within the lens housing 329. An output fiber bundle 339 may have a polarizer 331 and may couple into the objective lens 350.

As an overview of the example, the illumination system 312 provides illumination 340 that illuminates the eye 316. The laser system 320 provides a laser beam. The dichroic mirror 324 directs the laser beam through the objective lens 326. The laser beam exits the laser head 328 through the patient interface 314 to the eye 316. The camera 322 generates an image of the eye 316.

Turning to the components, the illumination system 312 provides illumination 340 that illuminates the eye 316. In the embodiments, the light sources 332 (332a and 332b) provide light of any suitable wavelength, e.g., ultraviolet (10 nanometers (nm) to 400 nm), visible (400 to 700 nm), and/or infrared (700 nm to 1 millimeter (mm), e.g., 700 to 800 nm, 800 to 900 nm, and/or 900 nm to 1 mm). The light sources may provide light of the same wavelength or may provide light of different wavelengths. For example, one light source 332 (332a or 332b) may provide a visible light, and the other light source 332 (332b or 332a, respectively) may provide an infrared light. Examples of light sources 332 (332a and 332b) include light-emitting diode (LED), such as an LED chip. While two light sources are illustrated, it will be appreciated that any number of light sources may be used, including three, four, five, six, or more distinct light sources. Additionally or alternatively, a given light source may be configurable such that two light sources may provide different combinations of wavelengths of illumination.

A light router 336 receives light from the light sources 332 (332a and 332b) via input fibers 334 (334a and 334b). A light router 336 may be any suitable device that can route light from the light sources 332 (332a and 332b) to the output fibers 338. For example, the light router 336 may be a light randomizer that receives from the light sources 332 (332a and 332b) via optical fibers. As another example, the light router 336 may be a light engine that receives light from the light sources 332 (332a and 332b) via light paths.

The input fibers 334 (334a and 334b) and output fibers 338 may include any suitable optical fibers. Examples of optical fibers include borosilicate glass fibers. The optical fibers may have any suitable size, e.g., in the range of 20 to 80 microns, such as 20 to 40 microns, 40 to 60 microns, and/or 60 to 80 microns. The optical fibers may have any suitable numerical aperture, e.g., in the range of 0.5 to 0.7, such as 0.5 to 0.6 and/or 0.6 to 0.7. The optical fibers may be arranged in any suitable manner. For example, the input fibers 334 (334a and/or 334b) and/or output fibers 338 may be arranged in fiber bundles of any suitable diameter in a range of, e.g., 0.2 to 3 millimeters (mm), such as 0.2 to 0.5 mm, 0.5 to 1 mm, 1 to 1.5 mm, 1.5 to 2 mm, and/or 2 to 3 mm. Examples of fiber bundles are described herein.

The output fibers 338 direct the light from the light sources 332 (332a and 332b) via the light router 336 and the input fibers 334 towards the patient interface 314 to provide the illumination 340 that illuminates the eye 316. The output fibers 338 may include any suitable number of fibers in a range of, e.g., 1 to 1000 fibers, 1000 to 2000 fibers, 2000 to 3000 fibers, 3000 to 4000 fibers, 4000 to 5000 fibers, and/or greater than 5000 fibers. The output fibers 338 may comprise optical fibers that provide the illumination 340 in any suitable manner. The optical fibers may be arranged individually and/or may be grouped into output fiber bundles 339. An output fiber bundle 339 may have any suitable number of fibers, e.g., 1 fiber to 500 fibers, 500 to 1000 fibers, 1000 to 1500 fibers, 1500 to 2000 fibers, and/or greater than 2000 fibers. The output fiber bundles 339 may each have the same number of fibers or may have different numbers of fibers.

The output fibers 338 and/or output fiber bundles 339 may be dispersed about the lens housing 329 in any suitable manner. For example, the output fibers 338 and/or output fiber bundles 339 may be dispersed around the outer surface of the lens housing 329 and/or within the inner surface 362 of the lens housing 329. As another example, the output fibers 338 and/or output fiber bundles 339 may be arranged equidistant from each other and/or may be arranged at different distances from each other, e.g., the output fibers 338 and/or output fiber bundles 339 may be arranged closer together where more illumination may be needed. In some embodiments, the number and arrangement of fibers may provide the appearance or perception of an entire circumference of illumination.

In one or more embodiments, the output fibers 338 may include any suitable number of polarizers 331, e.g., at least one polarizer 331 for at least a subset of the fiber bundles. In the example, each output fiber bundle 339 has a polarizer 331. In other examples, one or more fiber bundles may lack a polarizer 331. A polarizer 331 may be located at any suitable location relative to the output fiber 338. In the example, the polarizer 331 is located at the end of the output fiber bundle 339 and disposed between the output fiber bundle 339 and the objective lens 350. The polarizer 331 may operate to polarize the illumination exiting the associated output fiber bundle 339, e.g., by attenuating or otherwise removing any illumination not aligned with the polarity of the polarizer 331.

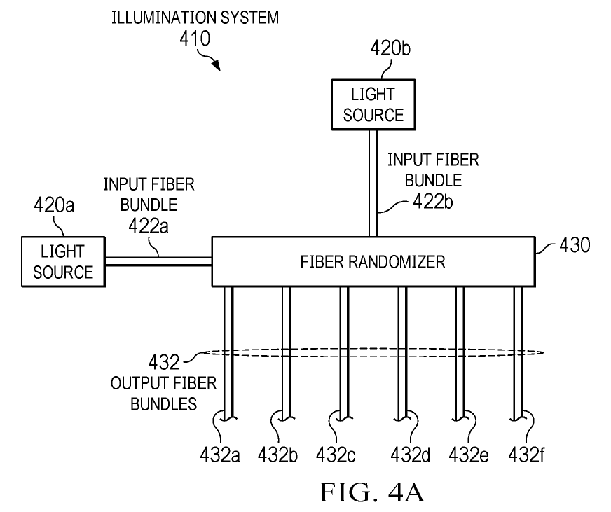

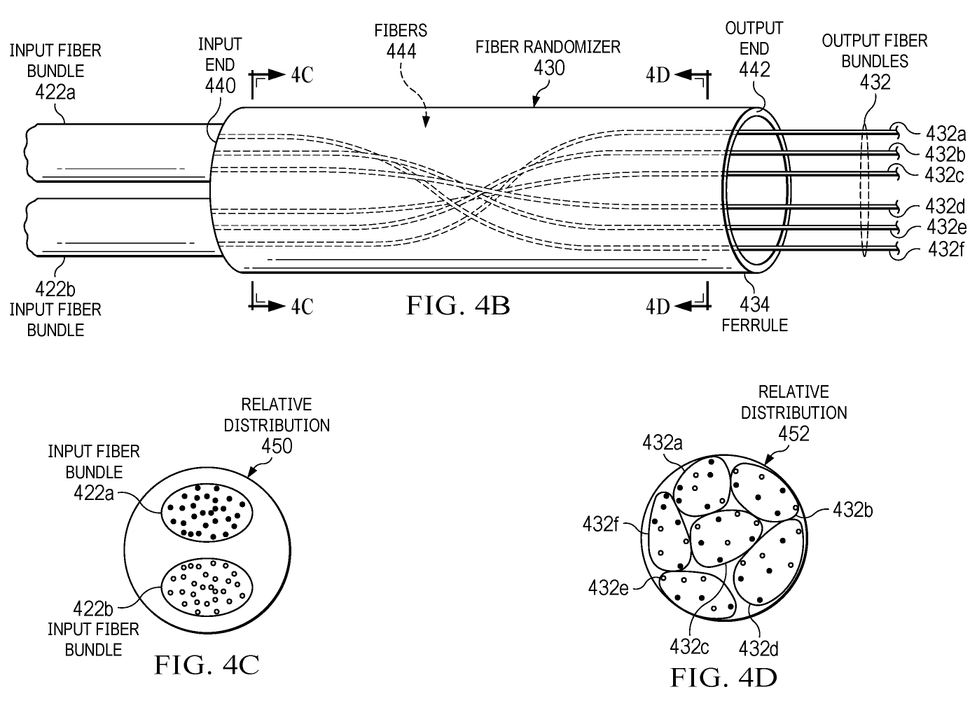

FIGS. 4A through 4D illustrate an example of an illumination system 410 with a fiber randomizer 430, according to at least one embodiment described in the present disclosure. FIG. 4A shows the illumination system 410 that includes light sources 420 (420a and 420b), input fiber bundles 422 (422a and 422b), the fiber randomizer 430, and/or output fiber bundles 432 (432a to 432f), which may be coupled as shown. FIG. 4B shows a simplified diagram of the fiber randomizer 430, which comprises a ferrule 434 (with an input end 440 and an output end 442) through which fibers 444 pass. The input fiber bundles 422 (422a and 422b) go into the input end 440, and output fiber bundles 432 (432a to 432f) exit via the output end 442. FIG. 4C shows an illustrative example of relative distribution 450 of the input fiber bundles 422 (422a and 422b) at the input end 440. FIG. 4D shows an illustrative example of relative distribution 452 of the fibers 444 at the output end 442. Please note that the relative distribution 450 and the relative distribution 452 are simplified descriptions of the distributions of fibers, and the dots that represent the fibers are not intended to indicate the specific number of fibers.

Turning to the components, a light source 420 (420a or 420b) may be any suitable light source, e.g., a LED light source, that provides any suitable light, as described herein. For example, a light source 420a or 420b provide a visible light, and the other light source 420b or 420a, respectively, may provide an infrared light. The input fiber bundles 422 (422a and 422b) receive light from the light sources 420 (420a and 420b, respectively) and delivers the light to the input end 440 of the fiber randomizer 430.

The fiber randomizer 430 changes the relative distribution 450 of the fibers 444 at the input end 440 to the relative distribution 452 of fibers 444 at the output end 442. In the example, the fibers 444 of the input fiber bundles 422 (422a and 422b) at the input end 440 have the relative distribution 450 where the fibers of the input fiber bundle 422a are grouped together and the fibers of the input fiber bundle 422b are grouped together. The fiber randomizer 430 rearranges the relative position of the fibers 444 to create the relative distribution 452 where the fibers of the input fiber bundle 422a are randomly dispersed relative to the fibers of the input fiber bundle 422b, i.e., where there is a randomized distribution of the fibers of the input fiber bundle 422a and the fibers of the input fiber bundle 422b.

The relative distribution 452 may be created in any suitable manner. In one or more embodiments, the relative distribution 452 may be created by interweaving the fibers of the input fiber bundle 422a and the fibers of the input fiber bundle 422b along a length of the fiber randomizer 430. Interweaving fibers may include interlacing and/or interspersing fibers. For example, the fiber randomizer 430 may comprise the fibers of the input fiber bundle 422a interlaced with the fibers of the input fiber bundle 422b to yield a randomized distribution of the fibers of the input fiber bundle 422a and the fibers of the input fiber bundle 422b.

The fiber randomizer 430 may change the relative distribution 450 in any suitable manner. For example, if there are a number P input fiber bundles 422, then the maximum percentage of fibers from a particular input bundle 422 that appears in a particular output fiber bundle 432 may be in a range of, e.g., (1/P)100% to 100%. In the example with input fiber bundles 422a and 422b, the maximum percentage of fibers from the input fiber bundle 422a that is in any of the output fiber bundles 432 may be in a range of, e.g., 50% to 100%, such as 50% to 65%, 65% to 80%, and/or 80% to 100%. In one or more embodiments, one or more output fiber bundles 432 may include, e.g., approximately 50% fibers from the input fiber bundle 422a and approximately 50% fibers from the input fiber bundle 422b, i.e., an approximately equal number of fibers from the input fiber bundle 422a and fibers from the input fiber bundle 422b. An output fiber bundle 432 may have any suitable relative distribution 452. For example, the relative distribution 452 may be 0 to 20 percent, 20 to 30 percent, 30 to 40 percent, and/or 40 to 50 percent of fibers from one input fiber bundle 422a or 422b, and 100 to 80 percent, 80 to 70 percent, 70 to 60 percent, and/or 60 to 50 percent, respectively, of fibers from the one input fiber bundle 422b or 422a, respectively.

While described as receiving and distributing fibers, it will be appreciated that the fiber randomizer 430 may include a mechanical component that is fixed in orientation, shape, and size and may not be a component that is continuously, ad hoc, or repeatedly readjusting or rearranging its internal fibers after manufacturing. For example, the placement of the fibers and their orientation and distribution of the fibers within the fiber randomizer 430 during manufacturing may be the receiving and distributing of the fibers that result in the randomized distribution of the fibers from one end of the fiber randomizer 430 to the other.

The output fiber bundles 432 may include any suitable number of output fiber bundles, e.g., 1, 2, 3, 4, 5, 6 to 8, 8 to 10, 10 to 15, or 15 or more bundles. In the example, the output fiber bundles 432 comprise six output fiber bundles 432a to 432f. In one or more embodiments, the output fibers may be fanned out and spread around the patient interface, as described in more detail herein.

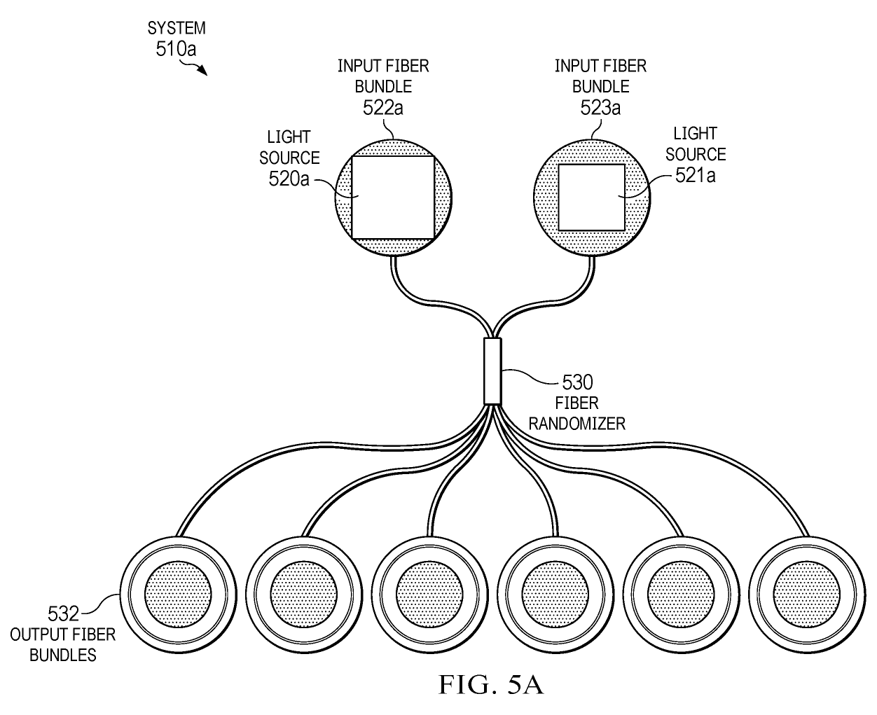

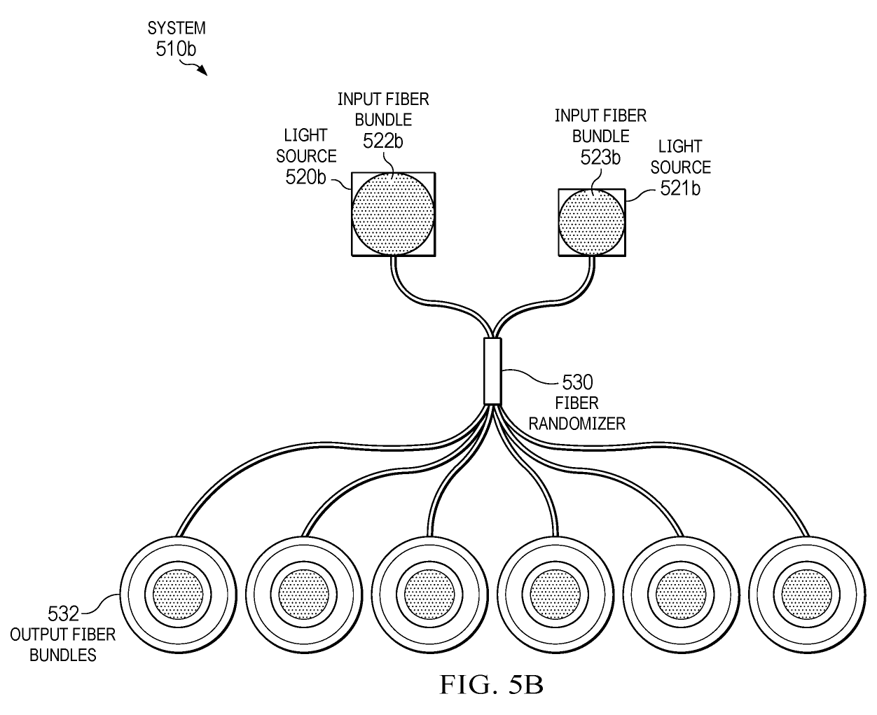

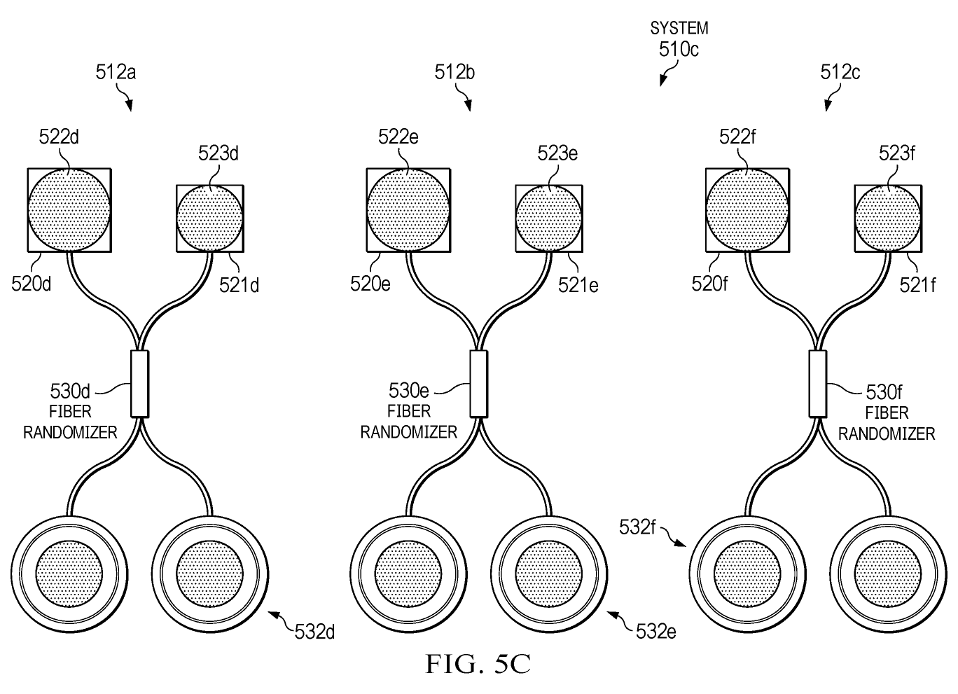

FIGS. 5A through 5C illustrate examples of illumination systems 510 (510a, 510b, and/or 510c) that include a fiber randomizer 530, according to at least one embodiment described in the present disclosure. FIGS. 5A and 5B illustrate examples of illumination systems 510 (510a and/or 510b) with a 2x6 fiber bundle. In the examples, an illumination system 510 (510a or 510b) includes a light source 520 (520a and/or 520b, respectively), a light source 521 (521a and/or 521b, respectively), an input fiber bundle 522 (522a and/or 522b, respectively), an input fiber bundle 523 (523a and/or 523b, respectively), a fiber randomizer 530, and/or output fiber bundles 532.

As an overview of an example of the illumination system 510, an input fiber bundle 522 includes first fibers, where each first fiber can transmit a first light from the light source 520, and an input fiber bundle 523 includes second fibers, where each second fiber can transmit a second light from the light source 521. Each output fiber bundle 532 includes a subset of output fibers and can provide both the first light and the second light to a patient interface. The fiber randomizer 530 optically couples the input fiber bundles 522 and 523 to the output fiber bundles 532.

Turning to the components, the light source 520 (520a and/or 520b) and the light source 521 (521a and/or 521b) may be as described herein. For example, the light source 520 (520a and/or 520b) may provide a visible light, and the light source 521 (521a and/or 521b) may provide an infrared light. The input fiber bundle 522 (522a and/or 522b) and the input fiber bundle 523 (523a and/or 523b) may be as described herein. For example, the input fiber bundle 522 (522a and/or 522b) and/or the input fiber bundle 523 (523a and/or 523b) may have a diameter in the range of 0.9 to 2 millimeters (mm), e.g., 0.9 to 1.2 mm, 1.2 to 1.6 mm, and/or 1.6 to 2 mm. The fiber randomizer 530 may be as described herein, e.g., may be a 2 x 6 fiber combiner / splitter. The output fiber bundles 532 may be as described herein. For example, the output fiber bundles 532 may have a diameter that is smaller than the diameter of an input fiber bundle, such as a diameter in the range of 0.4 to 1.2 millimeters (mm), such as 0.4 to 0.8 mm and/or 0.8 to 1.2 mm.

In the illustrated examples, the diameters of the input fiber bundle 522 (522a and/or 522b) and/or the input fiber bundle 523 (523a and/or 523b) may be larger than, smaller than, or equivalent to a dimension of the light source 520 (520a and/or 520b) and/or the light source 521 (521a and/or 521b), respectively, that provides light to the bundle. For example, in system 510a of FIG. 5A, the diameters of the input fiber bundle 522a and 523a are larger than a length of the side of the light source 520a and 521a, respectively. As another example, in system 510b of FIG. 5B, the diameters of the input fiber bundle 522b and 523b are substantially equivalent to the length of the side of the light source 520b and 521b, respectively.

FIG. 5C illustrates an example of an illumination system 510c with one or more 2x2 illumination subsystems 512 (512a, 512b, and/or 512c). In the example, an illumination subsystem 512 (512a, 512b, and/or 512c) includes a first light source 520 (520d, 520e, and/or 520f, respectively), a second light source 521 (521d, 521e, and/or 521f, respectively), a first input fiber bundle 522 (522d, 522e, and/or 522f, respectively), a second input fiber bundle 523 (523d, 523e, and/or 523f, respectively), a fiber randomizer 530 (530d, 530e, and/or 530f, respectively), and/or output fiber bundles 532 (532d, 532e, and/or 532f, respectively).

Turning to the components, the first light source 520 (520d, 520e, and/or 520f) and the second light source 521 (521d, 521e, and/or 521f) may be as described herein. For example, the first light source 520 (520d, 520e, and/or 520f) may provide a visible light, and the second light source 521 (521d, 521e, and/or 521f) may provide an infrared light. The first input fiber bundle 522 (522d, 522e, and/or 522f) and the second input fiber bundle 523 (523d, 523e, and/or 523f) may be as described herein. For example, the first input fiber bundle 522 (522d, 522e, and/or 522f) and/or the second input fiber bundle 523 (523d, 523e, and/or 523f) may have a diameter in the range of 0.9 millimeters (mm) to 1.2 mm. The fiber randomizer 530 (530d, 530e, and/or 530f) may be as described herein, e.g., may be a 2 x 2 fiber combiner / splitter. The output fiber bundles 532 (532d, 532e, and/or 532f) may be as described herein. For example, the output fiber bundles 532 may have a diameter that is smaller than the diameter of an input fiber bundle, such as a diameter in the range of 0.9 millimeters (mm) to 1.2 mm.

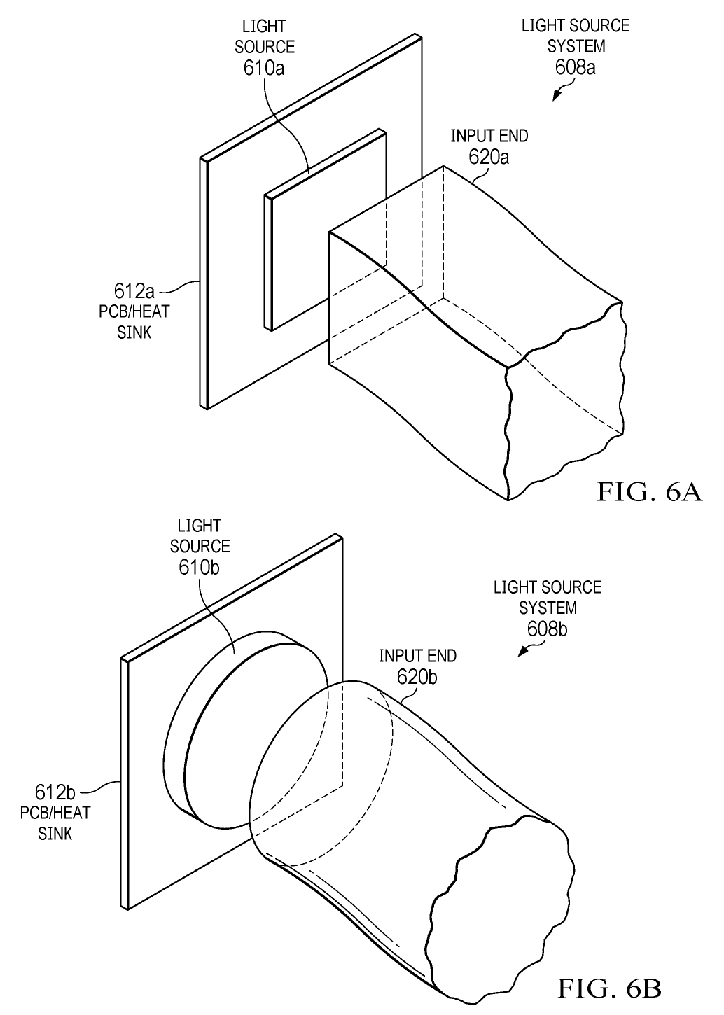

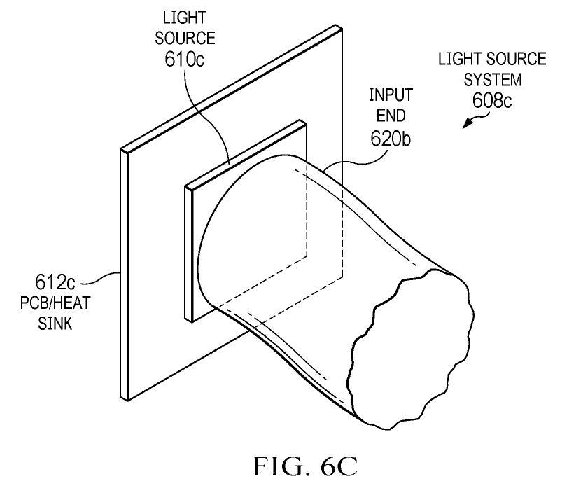

FIGS. 6A through 6C illustrate examples of input ends 620 (620a, 620b, and/or 620c) that receive light from light source systems 608 (608a, 608b, and/or 608c, respectively), according to at least one embodiment described in the present disclosure. A light source system 608 (608a, 608b, and/or 608c) includes a light source 610 (610a, 610b, and/or 610c respectively) and a printed circuit board (PCB) / heat sink 612 (612a, 612b, and/or 612crespectively).

Turning to the components, the light source system 608 (608a, 608b, and/or 608c) provides light to the input ends 620 (620a, 620b, and/or 620c, respectively). A light source 610 (610a, 610b, and/or 610c) may be any suitable light source, which may be as described herein. In the examples, the light source 610 (610a, 610b, and/or 610c) may be an LED chip with a square shape with sides in the range of 0.5 to 1.5 nanometers (nm), such as 0.5 to 0.8 nm, 0.8 to 1 nm, 1 to 1.2 nm, and/or 1.2 to 1.5 nm. The PCB / heat sink 612 (612a, 612b, and/or 612c) may include a PCB and/or a heat sink.

An input end 620 may have any suitable shape or size. For example, a cross-section (which may be orthogonal to the fiber axis) of an input end 620 may have a shape and/or size that matches (e.g., is similar to or the same as) the shape and/or size of the light source 610, which may optimize the amount of light received by the input end 620. In one or more embodiments, the fibers of an input end 620 may be fused, which may improve light transmission. In one or more embodiments, there may be an air gap between input ends 620 (620a, 620b, and/or 620c) and the light source 610 (610a, 610b, and/or 610c respectively). The air gap may have any suitable size, e.g., a value between 0.01 to 6 millimeters (mm), such as 0.01 to 0.1 mm, 0.1 to 1 mm, 1 to 2 mm, 2 to 4 mm, and/or 4 to 6 mm.

In FIG. 6A, the input fiber end 620a receives light from the light source 610a. The light source 610a has a square shape. In the example, the cross-section of the input fiber end 620a also has a square shape. In other examples, the cross-section of the input fiber end 620a may have a different shape, e.g., a circular shape.

In FIG. 6B, the input fiber end 620b receives light from the light source 610b. The light source 610b has a circular shape. In the example, the cross-section of the input fiber end 620a also has a circular shape. In other examples, the cross-section of the input fiber end 620a may have a different shape, e.g., a square shape.

In FIG. 6C, the input fiber end 620c receives light from the light source 610c. The light source 610c has a square shape. In the example, the cross-section of the input fiber end 620a has a circular shape. In other examples, the cross-section of the input fiber end 620a may have a different shape, e.g., a square shape.

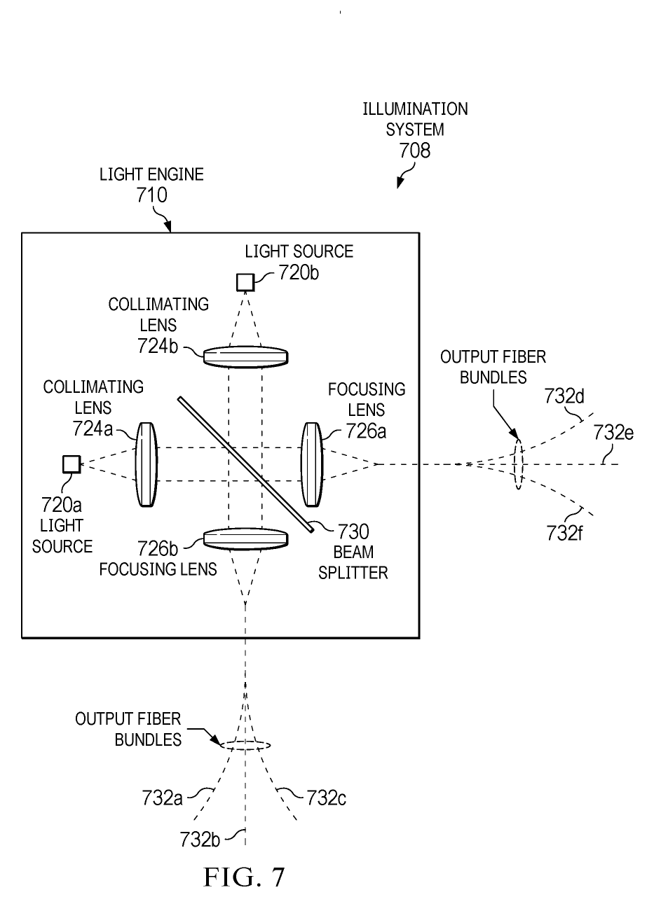

FIG. 7 illustrates an example of an illumination system 708 with a light engine 710, according to at least one embodiment described in the present disclosure. In the example, the illumination system 708 includes the light engine 710 and/or output fiber bundles 732 (732a to 732f), which may be coupled as shown. The light engine 710 includes light sources 720 (720a and/or 720b), collimating lenses 724 (724a and/or 724b), focusing lenses 726 (726a and/or 726b), and/or a beam splitter 730, which may be coupled as shown.

As an overview of the example, the light engine 710 generates a first light using the light source 720a and a second light using the light source 720b. The light engine 710 splits the first light into first light portions and splits the second light into second light portions. Each output fiber bundle 732 (723a to 732f) receives a first light portion and a second light portion and provides the first light portion and the second light portion to illuminate the patient interface.

Turning to the components, a light source 720 (720a or 720b) may be any suitable light source, e.g., a LED light source, that provides any suitable light. In an example, one light source 720a or 720b may provide a visible light, and the other light source 720b or 720a, respectively, may provide an infrared light. The collimating lenses 724 (724a and 724b) collimate the light from the light sources 720 (720a and 720b, respectively) and direct the light to the beam splitter 730.

The beam splitter 730 splits the light from the light source 720a into a first portion for the focusing lens 726a and a second portion for the focusing lens 726b. Similarly, the beam splitter 730 splits the light from the light source 720b into a first portion for the focusing lens 726a and a second portion for the focusing lens 726b. Accordingly, each focusing lens 726a and 726b receives light from both light sources 720a and 720b. The beam splitter 730 may split the beam in any suitable manner, e.g., 10 to 20 percent, 20 to 30 percent, 30 to 40 percent, and/or 40 to 50 percent for the first portion and the remainder for the second portion (e.g., a 50:50 split, a 60:40 split, etc.). The focusing lenses 726a and 726b direct the light to the output fiber bundles 732d to 732f and 732a to 732c, respectively.

The output fiber bundles 732 may include any suitable number of output fiber bundles, e.g., 1, 2, 3, 4, 5, 6 to 8, 8 to 10, 10 to 15, or 15 or more bundles. In the example, the output fiber bundles 732 comprise six output fiber bundles 732a to 732f. In one or more embodiments, the output fibers may be fanned out and spread around the patient interface, as described in more detail herein.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

Additionally, relative terms may be used and understood as typically used in the relevant art. By way of example, relative terms such as “approximately,” “substantially,” “about,” “roughly,” or other similar relative terms mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When such relative terms are used to refer to numbers or values that are part of a range, the term can be used to modify both the minimum and maximum numbers or values.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed:

1. An illuminator system configured to provide illumination for a patient interface, the illuminator system comprising:
a plurality of input bundles comprising:
a first input bundle comprising a plurality of first fibers, each first fiber of the plurality of first fibers configured to transmit a first light from a first light source; and
a second input bundle comprising a plurality of second fibers, each second fiber of the plurality of second fibers configured to transmit a second light from a second light source of a different wavelength than the first light source;
a plurality of output bundles, each output bundle of the plurality of output bundles comprising a subset of a plurality of subsets of output fibers, the each output bundle of the plurality of output bundles configured to provide both the first light and the second light to the patient interface; and
a fiber randomizer configured to optically couple the plurality of input bundles and the plurality of output bundles.

2. The illuminator system of claim 1, the plurality of output bundles arranged to provide the first light and the second light around a circumference of the patient interface.

3. The illuminator system of claim 1, at least one output bundle of the plurality of output bundles coupled to an objective lens of an ophthalmic microscope.

4. The illuminator system of claim 1, at least one input bundle of the plurality of input bundles having a diameter in a range of 0.4 millimeters (mm) to 2 mm.

5. The illuminator system of claim 1, at least one output bundle of the plurality of output bundles having a diameter in a range of 0.4 millimeters (mm) to 2 mm.

6. The illuminator system of claim 1, a shape of an input end of the first input bundle substantially equivalent to a shape of the first light source.

7. The illuminator system of claim 1, a size of an input end of the first input bundle substantially equivalent to a size of the first light source.

8. The illuminator system of claim 1, a number of the plurality of output bundles being in a range of three to twelve bundles.

9. The illuminator system of claim 1, the first light comprising a visible light.

10. The illuminator system of claim 1, the second light comprising an infrared light.

11. The illuminator system of claim 1, the fiber randomizer comprising:
a randomizer input end having a first relative distribution of fibers comprising:
the plurality of first fibers arranged in the first input bundle; and
the plurality of second fibers arranged in the second input bundle; and
a randomizer output end having a second relative distribution of fibers arranged as the plurality of subsets of output fibers, each subset of the plurality of subsets of output fibers comprising at least one first fiber of the plurality of first fibers and at least one second fiber of the plurality of second fibers.

12. The illuminator system of claim 11, wherein the second relative distribution of fibers includes a randomized distribution of the plurality of first fibers and the plurality of second fibers.

13. The illuminator system of claim 11, wherein the second relative distribution of fibers is created by interweaving the plurality of first fibers and the plurality of second fibers along a length of the fiber randomizer.

14. The illuminator system of claim 11, wherein a given subset of the plurality of subsets of output fibers includes approximately an equal number of the first fibers and the second fibers.

15. An illuminator system configured to provide illumination for a patient interface, the illuminator system comprising:
a plurality of illumination subsystems, an illumination subsystem of the plurality of illumination subsystems comprising:
a plurality of input bundles comprising:
a first input bundle comprising a plurality of first fibers, each first fiber of the plurality of first fibers configured to transmit a first light from a first light source; and
a second input bundle comprising a plurality of second fibers, each second fiber of the plurality of second fibers configured to transmit a second light from a second light source of a different wavelength than the first light source;
a plurality of output bundles, each output bundle of the plurality of output bundles comprising a subset of a plurality of subsets of output fibers, the each output bundle of the plurality of output bundles configured to provide both the first light and the second light to the patient interface; and
a fiber randomizer configured to optically couple the plurality of input bundles and the plurality of output bundles; and
the plurality of illumination subsystems configured to provide the first light and the second light to the patient interface.

16. The illuminator system of claim 15, the plurality of illumination subsystems arranged to provide the first light and the second light around a circumference of the patient interface.

17. The illuminator system of claim 15, at least one output bundle of the plurality of output bundles coupled to an objective lens of an ophthalmic microscope.

18. The illuminator system of claim 15, at least one input bundle of the plurality of input bundles having a diameter in a range of 0.4 millimeters (mm) to 2 mm.

19. The illuminator system of claim 15, at least one output bundle of the plurality of output bundles having a diameter in a range of 0.4 millimeters (mm) to 2 mm.

20. The illuminator system of claim 15, a shape of an input end of the first input bundle substantially equivalent to a shape of the first light source.

21. The illuminator system of claim 15, a size of an input end of the first input bundle substantially equivalent to a size of the first light source.

22. The illuminator system of claim 15, a number of the plurality of output bundles being in a range of three to twelve bundles.

23. The illuminator system of claim 15, the first light comprising a visible light.

24. The illuminator system of claim 15, the second light comprising an infrared light.

25. The illuminator system of claim 15, the fiber randomizer comprising:
a randomizer input end having a first relative distribution of fibers comprising:
the plurality of first fibers arranged in the first input bundle; and
the plurality of second fibers arranged in the second input bundle; and
a randomizer output end having a second relative distribution of fibers arranged as the plurality of subsets of output fibers, each subset of the plurality of subsets of output fibers comprising at least one first fiber of the plurality of first fibers and at least one second fiber of the plurality of second fibers.

26. An illuminator system configured to provide illumination for a patient interface, the illuminator system comprising:
a light engine configured to:
generate a first light using a first light source;
generate a second light using a second light source;
split the first light into a plurality of first light portions; and
split the second light into a plurality of second light portions; and
a plurality of output bundles, each output bundle of the plurality of output bundles configured to:
receive a first light portion of the plurality of first light portions;
receive a second light portion of the plurality of second light portions; and
provide the first light portion and the second light portion to illuminate the patient interface.

27. The illuminator system of claim 26, the plurality of output bundles arranged to provide the first light and the second light around a circumference of the patient interface.

28. The illuminator system of claim 26, at least one output bundle of the plurality of output bundles coupled to an objective lens of an ophthalmic microscope.
